(19) United States
(12) Patent Application Publication
(10) Pub. No.: US 2018/0208525 A1
(43) Pub. Date: Jul. 26, 2018

(54) PROCESS FOR THE OXIDATIVE COUPLING OF METHANE

(71) Applicant: SHELL OIL COMPANY, HOUSTON, TX (US)

(72) Inventors: Ronald Van SCHOONEBEEK, Amsterdam (NL); Andrew David HORTON, Amsterdam (NL); Andrzej Aleksander PEKALSKI, Manchester (GB); Alouisius Nicolaas Renée BOS, Amsterdam (NL); Hendrik DATHE, Amsterdam (NL); Carolus Matthias Anna Maria MESTERS, Sugar Land, TX (US)

(21) Appl. No.: 15/745,149

(22) PCT Filed: Jul. 15, 2016
(86) PCT No.: PCT/EP2016/066883

§ 371 Date: Jan. 16, 2018

(30) Foreign Application Priority Data
Jul. 16, 2015 (EP) 15177151.6

(51) Int. Cl. C07C 2/84; B01J 21/08; B01J 23/34; B01J 23/30; B01J 23/04; B01J 35/04; B01J 19/24

(52) U.S. Cl. CPC C07C 2/84; B01J 21/08; B01J 23/34; B01J 23/30; B01J 23/04; B01J 35/04; B01J 2219/30475; C07C 2521/08; C07C 2523/04; C07C 2523/30; C07C 2523/34; B01J 2219/00051; B01J 19/2415

(57) ABSTRACT

The invention relates to a process for the oxidative coupling of methane comprising converting methane to one or more C2+ hydrocarbons in a reactor, wherein said process comprises contacting a reactor feed comprising methane and oxygen with a catalyst composition and wherein the linear gas velocity of said reactor feed in the region above the catalyst bed is at least 0.6 m/s, the linear gas velocity through the catalyst bed is at least 0.6 m/s and the partial pressure of oxygen in the reactor is greater than 0.08 MPa.

### FIELD OF THE INVENTION

The present invention relates to a process for the oxidative coupling of methane.

### BACKGROUND OF THE INVENTION

Methane is a valuable resource which is used not only as a fuel, but is also used in the synthesis of chemical compounds such as higher hydrocarbons.

The conversion of methane to other chemical compounds can take place via indirect conversion wherein methane is reformed to synthesis gas (hydrogen and carbon monoxide), followed by reaction of the synthesis gas in a Fischer-Tropsch process. However, such indirect conversion is costly and consumes a lot of energy.

Consequently, it is desirable for industry to be able to convert methane directly to other chemical compounds without requiring the formation of intermediates such as synthesis gas. To this end, there has been increasing focus in recent years on the development of processes for the oxidative coupling of methane (OCM).

The oxidative coupling of methane converts methane into saturated and unsaturated, non-aromatic hydrocarbons having 2 or more carbon atoms, including ethylene. In this process, a gas stream comprising methane is contacted with an OCM catalyst and with an oxidant, such as oxygen or air. In such a process, two methane molecules are first coupled into one ethane molecule, which is then dehydrogenated into ethylene. Said ethane and ethylene may further react into saturated and unsaturated hydrocarbons having 3 or more carbon atoms, including propane, propylene, butane, butene, etc. Therefore, usually, the gas stream leaving an OCM process contains a mixture of water, hydrogen, carbon monoxide, carbon dioxide, methane, ethane, ethylene, propane, propylene, butane, butene and saturated and unsaturated hydrocarbons having 5 or more carbon atoms.

In general, the conversion that can be achieved in an OCM process is relatively low. Besides, at a higher conversion, the selectivity decreases so that it is generally desired to keep the conversion low. As a result, a relatively large amount of unconverted methane leaves the OCM process. The proportion of unconverted methane in the OCM product gas stream may be as high as 50 to 60 mol % based on the total molar amount of the gas stream. This unconverted methane has to be recovered from the desired products, such as ethylene and other saturated and unsaturated hydrocarbons having 2 or more carbon atoms, which are also present in such gas streams.

A further difficulty with OCM processes is that a competing reaction that takes place is the oxidation of methane to carbon dioxide and water.

In view of the afore-mentioned issues, there has been a great deal of attention focussed on developing optimised OCM processes and catalysts for use therein in order to increasing selectivity to C2+ hydrocarbons at lower reaction temperatures.

In this regard, one of the best-performing catalysts that has been found to date in the OCM field comprises manganese, tungsten and sodium on a silica carrier. The oxidative coupling of methane in the presence of said catalyst is studied in Applied Catalysis A: General 343 (2008) 142-148, Applied Catalysis A: General 425-426 (2012) 53-61, Fuel 106 (2013) 851-857, US 2014/0080699 A1 and U.S. Pat. No. 6,596,912 B1.

In order to obtain favourable C2+ hydrocarbon selectivity, OCM processes are normally run at low reactor pressures and/or with high methane:oxygen ratios in the reactor feed, that is to say, for example, at total reactor pressures in the range of from 0.1 to 0.5 MPa and/or with methane:oxygen ratios in the reactor feed in the range of from 4:1 to 6:1.

However, such measures lead to larger reactors and higher compression and separation costs.

Ekstrom et al. discuss the effect of pressure on the oxidative coupling reaction of methane in Applied Catalysis 1990, 62, 253.

Said document details the results of OCM experiments carried out at reactor pressures of 1-6 bara (0.1-0.6 MPa) with mixtures of methane and oxygen (in ratios of 90:10 or 85:15).

The influence of linear gas velocity in OCM was compared in Ekstrom et al. under empty quartz tube conditions with the use of various OCM catalysts (Li/MgO, Sm₂O₃ and Sr/Sm₂O₃). The maximum pO2 studied was 0.6 bara (0.06 MPa) in experiments with total pressure of 6 bara (0.6 MPa) (10% O₂) performed with linear gas velocity of 1.30 m/s using the Sm₂O₃ or Sr/Sm₂O₃ catalyst.

Ekstrom et al. discloses that the effect of the blank reaction (empty tube), which leads to high COx selectivity in OCM, can be reduced at oxygen partial pressures up to 0.6 bara (0.06 MPa) by applying linear gas velocities in the reactor above the catalyst bed of up to 1.30 m/s. Said document is not concerned with oxygen partial pressures of greater than 0.6 bara (0.06 MPa) and neither is said document concerned with linear gas velocities through the catalyst bed.

Chou et al. cite Ekstrom et al. in Journal of Natural Gas Chemistry 2002, 11, 131 in their investigation of the “Oxidative Coupling of Methane over Na—Mn—W/SiO₂ Catalysts at Elevated Pressures”.

Chou et al. discloses unfavourable effects on the OCM reaction due to the use of elevated pressures can be overcome by increasing GHSV. In particular, said document indicates that in the presence of Na—Mn—W/SiO₂ catalysts there is an optimum GHSV for the OCM at elevated pressures, and that higher GHSV favours the formation of C3-C4 hydrocarbons.

Chou et al. is not concerned with linear gas velocities in the OCM reaction.

It is highly desirable in the OCM field to develop processes which can be operated commercially in smaller reactors and with lower compression and separation costs.

### SUMMARY OF THE INVENTION

The present invention has surprisingly found that the implementation of certain measures allow the OCM process to be run with improved C2+ hydrocarbon selectivity at higher pressure, in particular when said process is carried out in the presence of a catalyst composition comprising manganese, one or more alkali metals and tungsten on a carrier, thereby allowing reactor size and compression and separation costs to be reduced.

Accordingly, the present invention provides a process for the oxidative coupling of methane comprising converting methane to one or more C2+ hydrocarbons in a reactor, wherein said process comprises contacting a reactor feed comprising methane and oxygen with a catalyst composition and wherein the linear gas velocity of said reactor feed in the region above the catalyst bed is at least 0.6 m/s, the linear gas velocity through the catalyst bed is at least 0.6 m/s and the partial pressure of oxygen in the reactor is greater than 0.08 MPa.

### BRIEF DESCRIPTION OF THE DRAWINGS

FIG. 1 is a schematic diagram showing a typical reactor set-up for oxidative coupling of methane.

FIG. 2 shows the position of catalyst composition and solid quartz tubes inside a typical 2.2 mm I.D. quartz reactor.

FIG. 3 shows the temperature profile inside a typical quartz reactor at oven set point of 750° C. and the position of catalyst.

### DETAILED DESCRIPTION OF THE INVENTION

To facilitate an understanding of the present invention, it is useful to define certain terms relating to the oxidative coupling of methane and the associated catalyst performance.

As used herein, “methane (CH₄) conversion” means the mole fraction of methane converted to product(s).

“Cx selectivity” refers to the percentage of converted reactants that went to product(s) having carbon number x and “Cx+ selectivity” refers to the percentage of converted reactants that went to the specified product(s) having a carbon number x or more. Thus, “C2 selectivity” refers to the percentage of converted methane that formed ethane and ethylene. Similarly, “C2+ selectivity” means the percentage of converted methane that formed compounds having carbon numbers of 2 or more.

“Cx yield” is used to define the percentage of products obtained with carbon number x relative to the theoretical maximum product obtainable. The Cx yield is calculated by dividing the amount of obtained product having carbon number x in moles by the theoretical yield in moles and multiplying the result by 100. “C2 yield” refers to the total combined yield of ethane and ethylene. The Cx yield may be calculated by multiplying the methane conversion by the Cx selectivity.

In reactors used for the oxidative coupling of methane, the reactor tubes are not typically completely filled with catalyst. Rather, the catalyst bed is typically located at some intermediate point in the catalyst tube. The reactor feed enters the reactor at a point above or upstream of the catalyst bed and passes through a region above the catalyst bed before passing through the catalyst bed.

As used herein, the “region above the catalyst bed” defines the section of the reactor located between the reactor inlet and the catalyst bed. It will be appreciated that in this context “above” is used to refer to the region upstream of the catalyst bed in the reactor. In some embodiments, however, where the reactor is in a vertical arrangement, the region “above” the catalyst bed may be vertically above the catalyst bed, as shown in FIG. 2.

In one embodiment of the present invention, the section of the reactor above the catalyst bed comprises inert porous packing, such that the linear gas velocity is increased relative to the empty reactor, without introducing a large pressure drop across this section of reactor. Examples of inert porous packings that may be conveniently applied in industrial reactors include one or more of foams, honeycombs, monoliths, balls and other forms of structured packing. In a preferred embodiment for application in laboratory testing, in addition to or as an alternative to the above packings, a solid insert may be applied. More preferably, said solid insert comprises quartz.

In the present invention, the linear gas velocity (in m/s) of the reactor feed comprising methane and oxygen—in the region above the catalyst bed is defined as being the total volume of the gas that passes 1 m² of open area above the catalyst bed per second. In formula: v=Qv/(eps*A), where Qv is the total gas flow rate, A the cross sectional area of the reactor just above the catalyst bed (regardless any internals or packings present) and eps the void fraction being the volume of area open for gas flow per volume of reactor volume (locally above the catalyst bed). The linear gas velocity may also be referred to as the interstitial velocity. This open area may be the annular area formed by inserting in solid insert or the voids in case of inert packings. In case of micro porous packings or structures, only reference is made to the “voids” which are present between the particles or structures and not to any (micro) pores inside the solid phase.

In the present invention, the linear gas velocity (in m/s) of the reactor feed comprising methane and oxygen through the catalyst bed is defined as being the following quotient: the flow rate of the reactor feed/cross-sectional surface area of the reactor/void fraction in the catalyst bed.

The factors determining the linear gas velocity of the reactor feed in the afore-mentioned quotients are further explained below.

The “cross-sectional surface area of the reactor” (in square meters; m²) means the surface area of the cross-section of the reactor excluding that portion of said surface area which is taken up by the wall of the reactor or other non-porous elements (e.g. baffles, heat exchangers, plates, etc.). Said cross-section is obtained by (imaginarily) cross-secting the reactor in a direction which is perpendicular to the direction of the reactor length. Said cross-section is the cross-section at the entrance of the catalyst bed. For example, in a case wherein the reactor is cylindrical, because of which the cross-section is circular, said “cross-sectional surface area of the reactor” is determined by the formula [Πd²]/4, wherein Π is “pi”, a dimensionless constant having a value of about 3.14, and “d” is the inner diameter (I.D.) (in meters) of the cylindrical reactor.

The “flow rate of the reactor feed” means the flow rate (in cubic meters/second; m³/s) of the reactor feed comprising methane and oxygen. In case two or more reactor feed gas streams are fed to the reactor, for example one reactor feed gas stream comprising oxygen and another reactor feed gas stream comprising methane, then said “flow rate of the reactor feed” means the sum of the flow rates of all of the reactor feed gas streams fed to the reactor. This flow rate is measured at the entrance of the catalyst bed, which is the position inside the reactor at which the reactor feed comprising methane and oxygen is contacted with catalyst particles for the first time. This implies, for example, that said flow rate is measured at the temperature and reactor pressure that exist at said entrance of the catalyst bed.

The “void fraction in the catalyst bed” is defined as follows: void fraction in the catalyst bed (dimensionless)=volume of voids in the catalyst bed/total volume of the catalyst bed. Said “volume of voids in the catalyst bed” consists of the volume of voids between the particles in the catalyst bed and does not include the volume of any pores present inside those particles, as would be present inside porous particles.

In the present specification, in the context of the catalyst bed, the term “voids” is used to indicate the voids which are present between the (catalyst) particles, whereas the term “pores” is used to indicate any voids (the “pores”) which may be present inside the (catalyst) particles as in porous (catalyst) particles.

Said “total volume of the catalyst bed” means the total volume of the catalyst particles, any inert particles and the voids between the particles. For example, in a case wherein the reactor is cylindrical, said “total volume of the catalyst bed” may be determined as follows. Firstly, the height of the catalyst bed inside the rector is determined by measuring the height of the empty part of the reactor not containing the catalyst bed and the height of the empty part of the reactor containing the catalyst bed. The difference between the latter 2 heights is the height of the catalyst bed inside the rector. Secondly, using the latter height and the cross-sectional surface area of the reactor, in that portion of the reactor where the catalyst bed is present, said “total volume of the catalyst bed” can be measured.

Said “void fraction in the catalyst bed” is defined by the following quotient: density of the particles/density of the catalyst bed. As discussed above, said particles comprise catalyst particles and any inert particles.

Said “density of the catalyst bed” may be determined as follows. Firstly, the total volume of the catalyst bed is determined as described above. Secondly, the total weight of the catalyst bed is divided by said total volume of the catalyst bed, resulting in the density of the catalyst bed.

Said “density of the particles” takes into account the presence of any pores inside the particles. Said “density of the particles” is defined by the following quotient: total weight of the particles/total volume of the particles. In said “total volume of the particles”, the volume of any pores present inside the (porous) particles is included and the volume of the voids which are present between the particles is excluded.

Said “density of the particles” may be determined by any suitable method known to the skilled person. A suitable method comprises contacting the particulate catalyst (catalyst particles), which particulate catalyst is preferably porous, with mercury. In this method, the above-mentioned “total volume of the particles”, including the volume of any pores present inside the (porous) particles and excluding the volume of the voids which are present between the particles, is determined. In this method, the pressure is chosen such that said pores are not filled with mercury whereas said voids are filled with mercury when the porous, particulate catalyst is contacted with mercury. Suitably, said pressure is atmospheric pressure. This method involves measuring, at said pressure, the volume of mercury filling a container wherein no particulate catalyst has been placed and measuring the volume of mercury filling the same container wherein a particulate catalyst of a given weight has been placed. The difference between these two volumes is the above-mentioned “total volume of the particles”. Such method is described by Clyde Orr, Jr. in “Application of Mercury Penetration to Materials Analysis”, Powder Technology, 3 (1969/70), pages 117-123, the disclosure of which is incorporated herein by reference, more in particular the section “Density” at page 121.

Thus, in the present invention, the linear gas velocity of the reactor feed through the catalyst bed, as defined above, is expressed as m³ reactor feed gas/m² voids/second, which is the volume of the reactor feed gas that passes 1 m² of voids in the catalyst bed per second. As mentioned above, by said “voids” only reference is made to the voids which are present between the (catalyst) particles and not to any pores inside those particles.

The residence time (in s) of the reactor feed in the hot zone region of the reactor above the catalyst is defined as the length of the hot zone above the catalyst divided by the gas linear velocity above the catalyst. The residence time of the reactor feed in the catalyst is defined as the length of the catalyst bed divided by the gas linear velocity in the catalyst bed. The total residence time in the hot zone is given by the sum of these values.

As used herein in the context of catalyst dopants, “weight percent” refers to the ratio of the total weight of the carrier, the metal-containing dopant or the metal in the dopant to the total weight of the catalyst composition the catalyst. Said percentages are determined with respect to the weight of the total dry catalyst composition. Suitably, the weight of the total dry catalyst composition may be measured following drying for at least four hours at 120 to 150° C.

Percentages of metals from the metal-containing dopants in the catalyst composition may be determined by XRF as is known in the art. The metals content of catalyst composition may also be inferred or controlled via its synthesis.

The components of the catalyst composition are to be selected in an overall amount not to exceed 100 wt. %.

As used herein, the term “compound” refers to the combination of a particular element with one or more different elements by surface and/or chemical bonding, such as ionic and/or covalent and/or coordinate bonding.

The term “ion” or “ionic” refers to an electrically chemical charged moiety; “cation” or “cationic” being positive, “anion” or “anionic” being negative, and “oxyanion” or “oxyanionic” being a negatively charged moiety containing at least one oxygen atom in combination with another element (i.e., an oxygen-containing anion). It is understood that ions do not exist in vacuo, but are found in combination with charge-balancing counter ions when added.

The term “oxidic” refers to a charged or neutral species wherein an element in question is bound to oxygen and possibly one or more different elements by surface and/or chemical bonding, such as ionic and/or covalent and/or coordinate bonding. Thus, an oxidic compound is an oxygen-containing compound which also may be a mixed, double or complex surface oxide. Illustrative oxidic compounds include, but are not limited to, oxides (containing only oxygen as the second element), hydroxides, nitrates, sulfates, carboxylates, carbonates, bicarbonates, oxyhalides, etc. as well as surface species wherein the element in question is bound directly or indirectly to an oxygen either in the substrate or the surface.

In the present invention, the linear gas velocity of said reactor feed comprising methane and oxygen in the region above the catalyst bed is at least 0.6 m/s, preferably at least 0.65 m/s, more preferably in the range of from 0.65 to 100 m/s and most preferably in the range of from 0.7 to 80 m/s.

In the present invention, the linear gas velocity of said reactor feed comprising methane and oxygen through the catalyst bed is at least 0.6 m/s, preferably at least 0.65 m/s, more preferably in the range of from 0.65 to 100 m/s and most preferably in the range of from 0.7 to 80 m/s.

In the present invention, the partial pressure of oxygen in the reactor is preferably greater than 0.08 MPa, more preferably greater than 0.1 MPa.

In the present invention, the linear gas velocity of the reactor feed comprising methane and oxygen in the region above the catalyst bed is at least 0.6 m/s, preferably at least 0.65 m/s, more preferably in the range of from 0.65 to 100 m/s, even more preferably in the range of from 0.65 to 90 m/s and most preferably in the range of from 0.7 to 80 m/s; and the linear gas velocity of said reactor feed comprising methane and oxygen through the catalyst bed is at least 0.6 m/s, preferably at least 0.65 m/s, more preferably in the range of from 0.65 to 100 m/s, even more preferably in the range of from 0.65 to 90 m/s and most preferably in the range of from 0.7 to 80 m/s.

In a preferred embodiment of the present invention, the linear gas velocity of the reactor feed comprising methane and oxygen in the region above the catalyst bed and the linear gas velocity of said reactor feed comprising methane and oxygen through the catalyst bed are both in the range of from 0.65 to 100/s, even more preferably in the range of from 0.65 to 90 m/s and most preferably in the range of from 0.7 to 80 m/s.

During the oxidative coupling of methane, a reactor feed comprising methane and oxygen is introduced into the reactor.

As used herein, the term “reactor feed” is understood to refer to the totality of the gaseous stream at the inlet of the reactor. Thus, as will be appreciated by one skilled in the art, the reactor feed is often comprised of a combination of one or more gaseous stream(s), such as a methane stream, an oxygen stream, a recycle gas stream, a diluent stream, etc.

In a preferred embodiment of the present invention, methane and oxygen are added to the reactor as a mixed feed, that is to say, a feed wherein a methane and an oxygen stream have been mixed together prior to addition to reactor.

However, in another embodiment of the present invention, there is so-called “distributed delivery” of reactants, whereby oxygen is added, for example, at multiple points in the reactor to ensure low oxygen concentrations in the reactor.

In a preferred embodiment of the present invention, unreacted methane is separated from the reactor product stream and is recycled to the reactor. Preferably, said recycled methane gas stream is combined with the main methane and oxygen streams as part of the reactor feed prior to entry into the reactor.

Methane may be present in the reactor feed in a concentration of at least 35 mole-% and preferably at least 40 mole-%, relative to the total reactor feed. Similarly, methane may be present in the reactor feed in a concentration of at most 90 mole-%, preferably at most 85 mole-%, relative to the total reactor feed.

In some embodiments of the present invention, methane may be present in the reactor feed in a concentration in the range of from 35 to 90 mole-%, preferably in the range of from 40 to 85 mole-%, relative to the total reactor feed.

In general, the oxygen concentration in the reactor feed should be less than the concentration of oxygen that would form a flammable mixture at either the reactor inlet or the reactor outlet at the prevailing operating conditions. Often, in practice, the oxygen concentration in the reactor feed may be no greater than a pre-defined percentage (e.g., 95%, 90%, etc.) of oxygen that would form a flammable mixture at either the reactor inlet or the reactor outlet at the prevailing operating conditions.

Although the oxygen concentration in the reactor feed may vary over a wide range, the oxygen concentration in the reactor feed is preferably at least 7 mole-%, more preferably at least 10 mole-%, relative to the total reactor feed. Similarly, the oxygen concentration of the reactor feed is preferably at most 25 mole-%, more preferably at most 20 mole-%, relative to the total reactor feed.

In some embodiments, oxygen may be present in the reactor feed in a concentration in the range of from 7 to 25 mole-%, preferably in the range of from 10 to 20 mole-%, relative to the total reactor feed.

It is within the ability of one skilled in the art to determine a suitable concentration of oxygen to be included in the reactor feed, taking into consideration, for example, the overall composition of the reactor feed, along with the other operating conditions, such as pressure and temperature.

However, in a preferred embodiment, the methane:oxygen volume ratio in the process of the present invention is in the range of from 2/1 to 10/1, more preferably in the range of from 3/1 to 6/1.

The reactor feed may further comprise one or more of a diluent gas, minor components typically present in the methane feed stream (e.g. ethane, propane etc.) or the methane recycle stream (e.g. ethane, ethylene, propane, propylene, carbon monoxide, carbon dioxide, hydrogen and water). The diluent represents the balance of the feed gas and is an inert gas. Examples of suitable inert gases are nitrogen, argon or helium.

The order and manner in which the components of the reactor feed are combined prior to contacting with the catalyst composition is not limited, and they may be combined simultaneously or sequentially. However, as will be recognized by one skilled in the art, it may be desirable to combine certain components of the inlet feed gas in a specified order for safety reasons. For example, oxygen may be added to the inlet feed gas after the addition of a dilution gas for safety reasons. Similarly, as will be understood by one of skill in the art, the concentration of various feed components present in the inlet feed gas may be adjusted throughout the process, for example, to maintain a desired productivity, optimize the process, etc. Accordingly, the above-defined concentration ranges were selected to cover the widest possible variations in the composition of the reactor feed during normal operation.

Thus, in one embodiment of the present invention, one reactor feed gas stream comprising methane and oxygen may be fed to the reactor. Alternatively, in other embodiments of the present invention, two or more reactor feed gas streams may be fed to the reactor, which gas streams form a combined reactor feed gas stream inside the reactor. For example, one reactor feed gas stream comprising methane and another reactor feed gas stream comprising oxygen may be fed to the reactor separately. Said one reactor feed gas stream or multiple reactor feed gas streams may additionally comprise an inert gas, as further described below.

The process of the present invention comprises utilising the catalyst composition in a reactor suitable for the oxidative coupling of methane.

The reactor may be any suitable reactor, such as a fixed bed reactor with axial or radial flow and with inter-stage cooling or a fluidized bed reactor equipped with internal and external heat exchangers.

As hereinbefore described, in one embodiment of the present invention, the catalyst composition may be packed along with an inert packing material, such as quartz, into a fixed bed reactor having an appropriate inner diameter and length.

Various reactor set-ups are described in the OCM field and the process of the present invention is not limited in that regard. The person skilled in the art may conveniently employ any of said reactor set-ups in conjunction with the process of the present invention.

Accordingly, reactor set-ups as described in EP 0206042 A1, U.S. Pat. No. 4,443,649 A, CA 2016675 A, U.S. Pat. No. 6,596,912 B1, US 2013/0023709 A1, WO 2008/134484 A2 and/or WO 2013/106771 A2 may be conveniently employed.

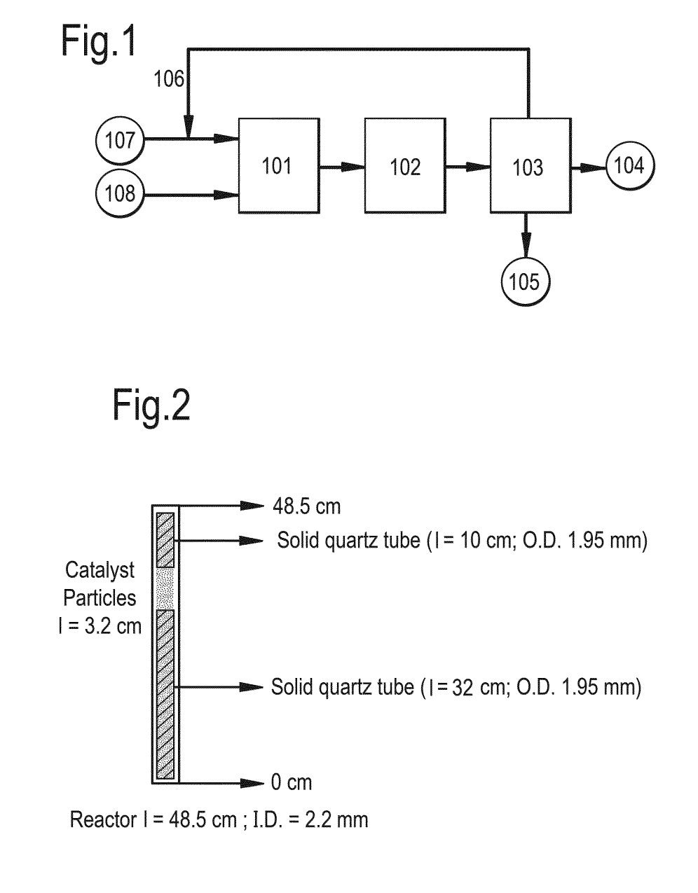

FIG. 1 is a schematic representation showing a typical reactor and product separation set-up for the oxidative coupling of methane.

Feed gas comprising methane and oxygen (or air) are introduced into the OCM reactor 101, via lines 107 and 108, respectively. The methane may consist of fresh feed and recycled methane (derived from the separation stage of the process). The product mixture exiting the OCM reactor is passed to condensation vessel 102, where the majority of the water by-product of OCM is removed. The product from 102 is then sent to the separation section 103, wherein the desired C2+ hydrocarbons are separated (stream 104), either as a mixed hydrocarbon stream or as separated streams of ethylene, ethane, propylene and other hydrocarbons. Unreacted methane separated from the OCM product mixture in 103 may optionally be recycled, as stream 106, which is combined with fresh feed stream 107, before entering the reactor. Undesired products of OCM, such as carbon monoxide and carbon dioxide, as well as nitrogen in the case of OCM with air feed, are also separated from the product mixture in 103 and leave the process as stream 105. The separation section may also include a section for conversion of alkanes to olefins (e.g. ethane cracker).

The process of the present invention is not limited to any particular reactor or flow configurations, and those depicted in FIG. 1 are merely exemplary. Furthermore, the sequence in which various feed components are introduced into the process and their respective points of introduction, as well as the flow connections, may be varied from that depicted in FIG. 1.

In the process of the present invention, the reactor feed comprising methane and oxygen is contacted with a catalyst composition in order to effect the conversion of methane to one or more C2+ hydrocarbons at a reactor temperature that is typically in the range of from 300 to 1000° C. Said conversion is effected at a reactor temperature preferably in the range from 400 to 900° C., more preferably in the range of from 650 to 850° C. and most preferably in the range of from 690 to 850° C.

The reactor temperature is defined as the feed temperature as measured just before the catalyst bed.

Preferably, the total pressure in the reactor is greater than 0.6 MPa, more preferably greater than 0.7 MPa and most preferably greater than 0.8 MPa.

In a preferred embodiment of the present invention, the conversion of methane to one or more C2+ hydrocarbons is effected at a total reactor pressure in the range of from 0.6 MPa to 1.4 MPa. More preferably, said reactor pressure is in the range of from 0.7 to 1.3 MPa, even more preferably in the range of from 0.8 to 1.2 MPa and most preferably in the range of from 0.9 to 1.1 MPa.

The gas hourly space velocity (GHSV) in the process of the present invention is the entering volumetric flow rate of the reactor feed (at standard conditions) divided by the catalyst bed volume. Preferably, said gas hourly space velocity is in the range of from 10000 to 400000 h⁻¹ and more preferably in the range of from 30000× to 300000 h⁻¹. Said GHSV is measured at standard temperature and pressure, namely 32° F. (0° C.) and 1 bara (100 kPa).

In general, the product stream comprises water in addition to the desired product. Water may easily be separated from said product stream, for example by cooling down the product stream from the reaction temperature to a lower temperature, for example room temperature, so that the water condenses and can then be separated from the product stream.

In a preferred embodiment, the process of the present invention has a C2+ hydrocarbon selectivity of greater than 45%, more preferably greater than 65%.

In a preferred embodiment, the process of the present invention results in an ethane:ethene mole ratio of less than 1.0, more preferably less than 0.5.

Preferably, the afore-mentioned C2+ hydrocarbon selectivity and ethane:ethene ratio values are determined at a reactor temperature in the range of from 650 to 850° C. and more preferably in the range of from 690 to 850° C.

The catalyst composition for use in the process of the present invention is not particularly limited and any catalyst that is known to be effective in catalyzing the oxidative coupling of methane may be conveniently employed.

Examples of such catalyst compositions includes those disclosed in WO 2008/134484 A2, U.S. Pat. No. 4,769,508 A, US 2013/0178680 A1, U.S. Pat. No. 6,596,912 B1, EP 0316075 A1, EP 0206042 A1, US 2013/0023709 A1, CA 2016675 A1, US 2014/0080699 A1, U.S. Pat. No. 6,576,803 and US 2010/0331595 A.

In a preferred embodiment of the present invention, the catalyst composition comprises manganese, one or more alkali metals and tungsten on a carrier.

The carrier is not limited and may be conveniently selected from one or more of silicon-, titanium-, zirconium- and aluminium-containing carriers such as silica, titania, zirconia and alumina.

The B.E.T. surface area, total pore volume, median pore diameter and pore size distribution of said carriers may be conveniently selected by the person skilled in the art.

The carrier may be present in the catalyst composition in an amount in the range of from 80-98% by weight, and most preferably in the range of from 92-96% by weight, relative to the total weight of the catalyst composition.

Typically, the preferred catalyst composition for use in the process of the present invention comprises manganese in an amount of in the range of from 1.0 to 10.0% by weight, preferably in the range of from 1.0 to 5.0% by weight, more preferably in the range of from 1.3 to 3.0% by weight and most preferably in the range of from 1.7 to 2.5% by weight, relative to the total weight of the catalyst composition.

In a preferred embodiment, the manganese is present in the catalyst composition in the form of one or more manganese-containing dopants such as one or more manganese-containing oxides. Said manganese-containing oxides may be reducible oxides of manganese and/or reduced oxides of manganese. However, in the active state, the catalyst composition comprises at least one reducible oxide of manganese. Such reducible oxides include compounds of the general formula MnₓO_{y} wherein x and y designate the relative atomic proportions of manganese and oxygen in the composition and one or more oxygen-containing Mn compounds which contain manganese, oxygen and additional elements. Particularly preferred reducible oxides of manganese include MnO₂, Mn₂O₃, Mn₃O₄ and mixtures thereof.

The preferred catalyst composition for use in the process of the present invention comprises one or more (Group 1) alkali metals. Said alkali metals are preferably from selected one or more of lithium, sodium, potassium, rubidium and cesium. Particularly preferred alkali metals are lithium and sodium.

The one or more alkali metals are preferably in a total amount of in the range of from 0.1 to 1.5% by weight, more preferably in the range of from 0.3 to 0.9% by weight, relative to the total weight of the catalyst composition.

The preferred catalyst composition for use in the process of the present invention further comprises tungsten. Said tungsten may be present in an amount of in the range of from 1 to 5% by weight, more preferably in the range of from 1.2 to 4.0% by weight, relative to the total weight of the catalyst composition.

A preferred catalyst composition for use in the process of the present invention comprises manganese, sodium and tungsten on a silica carrier.

In the preparation of the afore-mentioned preferred catalyst composition, the one or more alkali metals and tungsten may be doped as separate metals and/or metal-containing compounds into said composition. However, preferably, the one or more alkali metals and tungsten may be doped into the catalyst composition in the form of one or more compounds comprising both alkali metal(s) and tungsten therein. Suitable examples of such compounds include sodium tungstate and lithium tungstate.

During the oxidative coupling of methane according to the process of the present invention, the specific form of the manganese, one or more alkali metals, tungsten and any optional co-promoters and/or additional metal-containing dopants in the catalyst composition may be unknown.

Thus, when sodium, tungsten and manganese are present in combination in the catalyst composition, they may present as Na₂WO₄, Na₂W₂O₇ and/or Mn₂WO₄ and Mn₂O₃.

During the preparation of the afore-mentioned preferred catalyst composition, the specific form in which the manganese-containing dopant, the alkali metal-containing dopants, the tungsten-containing dopant and any optional co-promoters and/or additional metal-containing dopants are provided is not limited, and may include any of the wide variety of forms known.

For example, a manganese-containing dopant, an alkali metal-containing dopant, a tungsten-containing dopant and an optional co-promoter and/or additional metal-containing dopant may suitably be provided as ions (e.g., cation, anion, oxyanion, etc.), or as compounds (e.g., alkali metal salts, salts of a further co-promoter, etc.).

Generally, suitable compounds are those which can be solubilized in an appropriate solvent, such as a water-containing solvent.

As will be appreciated by persons skilled in the art, while specific forms of the afore-mentioned metal-containing dopants may be provided during catalyst preparation, it is possible that during the conditions of preparation of the catalyst composition and/or during use in oxidative coupling of methane, the particular forms initially present may be converted to other forms. Furthermore, in many instances, analytical techniques may not be sufficient to precisely identify the forms that are present. Accordingly, the afore-mentioned disclosure is not intended to be limited by the exact form of the manganese-containing dopant, the alkali metal-containing dopants, the tungsten-containing dopant and/or any optional co-promoters and/or additional metal-containing dopants that may ultimately exist on the catalyst composition during use.

Additionally, it should be understood that while a particular compound may be used during catalyst preparation (e.g., in an impregnation solution), it is possible that the counter ion added during catalyst preparation may not be present in the finished catalyst composition.

As previously discussed, the specific form in which the one or more alkali metals is provided is generally not limited, and may include any of the wide variety of forms known. For example, the one or more alkali metal-containing dopants may be provided as ions (e.g., cation), or as alkali metal compounds.

Examples of suitable alkali metal compounds include, but are not limited to, alkali metal salts and oxidic compounds of the alkali metals, such as the nitrates, nitrites, carbonates, bicarbonates, oxalates, carboxylic acid salts, hydroxides, halides, oxyhalides, borates, sulfates, sulfites, bisulfates, acetates, tartrates, lactates, oxides, peroxides, and iso-propoxides, etc.

As previously mentioned, the alkali metal-containing dopant may comprise a combination of two or more alkali metal dopants. Non-limiting examples include combinations of lithium and sodium, lithium and potassium, lithium and rubidium, lithium and cesium, sodium and potassium, sodium and rubidium, sodium and cesium, potassium and rubidium, potassium and cesium and rubidium and cesium.

Optionally, the preferred catalyst compositions for use in the process of the present invention may further comprise one or more co-promoters and/or additional metal-containing dopants.

Examples of co-promoters and metal-containing dopants that may be conveniently used therein include lanthanum, cerium, niobium and tin.

The catalyst composition may comprise said optional co-promoters and/or metal-containing dopants in a total amount of in the range of from 0.1 to 5% by weight, relative to the total weight of the catalyst composition.

Catalyst compositions for use in the process of the present invention may in principle be prepared by any suitable technique known in the art for similar catalyst compositions.

Thus, methods such as precipitation, co-precipitation, impregnation, granulation, spray-drying or dry-mixing can be used.

Optionally, prior to use in the process of the present invention, the catalyst composition may be pretreated at high temperature to remove moisture and impurities therefrom. Said pretreatment may take place, for example, at a temperature in the range of from 100-300° C. for about one hour in the presence of an inert gas such as helium.

The invention is further illustrated by the following Examples.

### EXAMPLES AND COMPARATIVE EXAMPLE

### Catalyst Preparation Procedure

### Preparation of Catalyst A

Catalyst A was prepared by impregnation of PQ silica. 1600 g PQ Silica (PD 11044, a commercial grade granular silica; 100-700 μm; surface area ca. 300 m²/g, pore volumes ca. 1.8 ml/g) was introduced into a rotating impregnation drum.

142.37 g Mn (NO₃)₂*4H₂O was dissolved in 2000 mL H₂O and 13.3 mL conc. HNO₃ (65%) was added to this solution. The final solution was made up to 2960 ml with H₂O.

This solution was added into the rotating drum (120 rpm) containing the afore-mentioned dried sample by a gear pump with a nozzle (nozzle distance 12 cm; silt nozzle 5, 2000 rpm). After the addition, the drum was rotated for 30 min at 20 rpm. The sample was then indirectly dried with a Leister fan for 45 minutes to drying grade >99.5.

34.97 g Na₂WO₄*2H₂O was dissolved in 2000 ml H₂O and 44.55 g citric acid monohydrate added. The final solution was made up to 2960 ml with H₂O. This solution was added into the rotating drum (120 rpm) containing the afore-mentioned dried sample by a gear pump with a nozzle (nozzle distance 12 cm, silt nozzle 5, 2000 rpm).

After the addition, the drum was rotated for 30 min at 20 rpm. The sample was then indirectly dried with a Leister fan for 45 minutes to drying grade 99.5 and afterwards calcined at 850° C. for 5 h applying a rate of increase of temperature of 3K/min.

### General Testing Procedure

Catalyst A was tested in a micro flow testing unit in accordance with the following general testing procedure.

A sieved fraction of Catalyst A (40-60 mesh), usually 50-60 mg (0.11 ml catalyst), was loaded in a 48.5 cm long quartz reactor with an internal diameter (I.D.) of 2.2 mm.

The catalyst composition was situated at the top part of the isothermal temperature profile of the reactor. Typically, the catalyst bed length was 3.2 cm. The reactor volume above and below the catalyst composition was filled up with a solid quartz tube having an outer diameter (O.D.) of 1.95 mm.

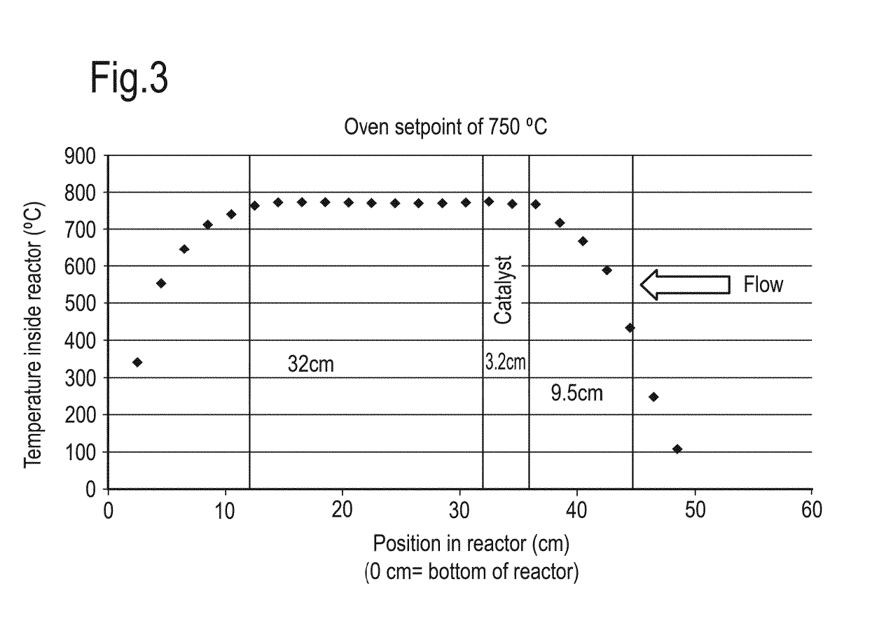

Schematic pictures of the isothermal profile, typical position of the catalyst composition and solid quartz tubes are given in FIGS. 2 and 3.

A reactor feed comprising of methane, oxygen and nitrogen was passed downflow over the catalyst composition being tested at a flow rate in the range of 5-11 Nl/hour (STP) and at a pressure in the range 0.2-1 MPa (2-10 bara).

The conversion of methane and oxygen was recorded at reactor temperatures in the range of 600-800° C. and the product composition was, after condensation of the water in a separator, measured with an on-line GC (Compact GC, Interscience, Breda) equipped with two TCD detectors and a FID detector for quantitative analyses of oxygen, nitrogen, carbon monoxide, carbon dioxide, methane, ethane, ethylene, C3, C4 and C5 hydrocarbons.

The total off-gas of the micro flow unit was determined by the amount of nitrogen (in Nl/hr) in the reactor feed and in the off gas (determined from the results of the on-line GC analyses). From this total off-gas flow, the individual component flows were calculated in Nl/hr. From this individual component flows, the total carbon balance was calculated and this was in most of the experiments between 98 and 102° C.

Besides the carbon balance, oxygen and methane conversions were calculated and also C2+ selectivity and yield.

For the purpose of calculating the linear gas velocity in the catalyst bed, the void fraction of the catalyst bed was assumed to be 0.4.

### Results

### Example 1

The OCM performance of Catalyst A was evaluated following the general procedure described above.

65.5 mg of Catalyst A was placed in a 48.5 cm long quartz reactor with an ID of 2.2 mm. The 3.2 cm catalyst bed was located in the top part of the isothermal temperature zone of the reactor (“hot zone”). Solid quartz tubes with OD of 1.95 mm were placed above and below the catalyst.

A mixture of methane (5.0 NL/h), oxygen (1.2 NL/h) and nitrogen (4.5 NL/h) at 0.98-1.0 MPa total pressure was passed down flow over the catalyst. The temperature was increased gradually from 700 to 760° C. and then back again to 740° C. GC analysis of the product mixture, as described above allowed the conversion of methane and oxygen and product selectivity and yield to be determined.

Key data for this experiment is detailed in Table 1.

### Example 2

This experiment was performed in a similar fashion to Example 1, again using 65.5 mg catalyst A, with the difference that the catalyst was placed towards the bottom of the “hot zone” (20 cm from top, compared to 1 cm in Example 1). Solid quartz tubes with OD of 1.95 mm were placed above and below the 3.2 cm catalyst bed.

In this Example, the temperature was increased gradually from 700 to 760° C. and then back again to 700° C.

Key data for this experiment is given in Table 1.

### Comparison Example 1

This experiment was performed with the same catalyst intake (65.5 mg) and gas feed composition and flow (10.7 NL/h) as used in Example 1.

A 3.1 mm ID quartz reactor was used instead of a 2.2 mm ID reactor and the catalyst (1.7 cm bed length) was placed in the center of the “hot zone” (10.6 cm below the top). A 3.0 mm OD quartz insert was placed below the catalyst, but no quartz insert was used above the catalyst.

The temperature was increased gradually from 700 to 760° C. and then back again to 700° C.

Key data for this experiment is given in Table 1.

### Comparison Example 2

This experiment was performed in a similar fashion to Comparison Example 1, except that the catalyst was placed at the top of the “hot zone” (1.3 cm from the top) rather than in the middle. Similar to Comparison Example 1, a 3.0 mm OD quartz insert was placed below the catalyst, but no quartz insert was used above the catalyst.

Key data for this experiment is given in Table 1.

As is summarized in Table 1 below, performed at comparable GHSV, the C2+ selectivity and yield in Examples 1 and 2 were found to be much higher than in Comparison Examples 1 and 2, demonstrating the surprising advantageous effects of the present invention that are obtained when higher gas linear velocities are applied both above the catalyst bed and through the catalyst bed and the OCM reaction is performed at high partial pressures of oxygen.

The residence time in the hot zone (region above the catalyst and in the catalyst) was as follows: Example 1, ca. 0.05 s; Example 2, ca. 0.2 s; Comparison Example 1 ca. 0.8 s; Comparison Example 2 ca. 0.14 s) Example 2 shows that high C2+ selectivity and yield may be obtained in high pressure OCM at “hot zone” residence times >0.1 s, provided that a high linear gas velocity is applied. Comparison Example 2 also illustrates that reducing the “hot zone” residence time does not lead to significantly enhanced selectivity when a low gas linear velocity is applied.

TABLE 1

| | | | | | | Linear | Linear | | | |
|---|---|---|---|---|---|---|---|---|---|---|
| | | | | | | gas | gas | | | |
| | | | | | | velocity | velocity | | | |
| | | | | | | above | through | | | |
| | Run | Pressure | Pressure | | | catalyst | catalyst | Conversion | Selectivity | Yield |
| | time | Total | O₂ | Temp | GHSV | bed | bed | CH₄ | O₂₊ | O₂₊ |
| Example | (h) | (MPa) | (MPa) | (° C.) | (h⁻¹) | (m/s) | (m/s) | (%) | (%) | (%) |
| Example | 2.0 | 0.98 | 0.11 | 700 | 88007 | 1.33 | 0.71 | 27.6 | 57.5 | 15.9 |
| 1 | 4.0 | 0.99 | 0.11 | 720 | 88007 | 1.35 | 0.72 | 26.7 | 54.9 | 14.7 |
| | 9.0 | 1.00 | 0.11 | 740 | 88007 | 1.36 | 0.73 | 26.5 | 53.2 | 14.1 |
| | 11.5 | 1.00 | 0.11 | 760 | 88007 | 1.38 | 0.74 | 26.6 | 52.1 | 13.9 |
| | 13.0 | 1.00 | 0.11 | 740 | 88007 | 1.35 | 0.73 | 26.7 | 53.5 | 14.3 |
| Example | 2.0 | 1.00 | 0.11 | 700 | 88007 | 1.30 | 0.70 | 28.9 | 57.0 | 16.5 |
| 2 | 6.0 | 1.00 | 0.11 | 720 | 88007 | 1.33 | 0.71 | 27.8 | 53.0 | 14.7 |
| | 7.5 | 1.00 | 0.11 | 740 | 88007 | 1.35 | 0.73 | 27.4 | 52.6 | 14.4 |
| | 10.0 | 1.00 | 0.11 | 760 | 88007 | 1.38 | 0.74 | 27.3 | 51.8 | 14.1 |
| | 13.0 | 1.00 | 0.11 | 740 | 88007 | 1.35 | 0.73 | 27.5 | 52.9 | 14.5 |
| | 16.0 | 1.00 | 0.11 | 720 | 88007 | 1.33 | 0.71 | 27.8 | 55.4 | 15.4 |
| | 20.0 | 1.00 | 0.11 | 700 | 88007 | 1.30 | 0.70 | 27.6 | 56.0 | 15.5 |
| Comp | 2.0 | 0.99 | 0.11 | 700 | 83434 | 0.14 | 0.35 | 23.9 | 27.8 | 6.6 |
| Example | 6.0 | 0.99 | 0.11 | 720 | 83434 | 0.14 | 0.36 | 24.0 | 28.4 | 6.8 |
| 1 | 9.0 | 0.99 | 0.11 | 740 | 83434 | 0.15 | 0.37 | 24.3 | 28.6 | 6.9 |
| | 11.5 | 0.99 | 0.11 | 760 | 83434 | 0.15 | 0.38 | 24.3 | 28.6 | 6.9 |
| | 14.0 | 0.99 | 0.11 | 740 | 83434 | 0.15 | 0.37 | 24.2 | 28.4 | 6.9 |
| | 18.0 | 0.99 | 0.11 | 720 | 83434 | 0.14 | 0.36 | 24.1 | 28.0 | 6.7 |
| | 21.5 | 0.99 | 0.11 | 700 | 83434 | 0.14 | 0.35 | 24.0 | 27.8 | 6.7 |
| Comp | 1.0 | 1.02 | 0.11 | 700 | 83434 | 0.14 | 0.34 | 24.1 | 29.3 | 7.1 |
| Example | 4.0 | 1.02 | 0.11 | 720 | 83434 | 0.14 | 0.35 | 24.3 | 30.1 | 7.3 |
| 2 | 7.0 | 1.02 | 0.11 | 740 | 83434 | 0.14 | 0.36 | 24.5 | 30.5 | 7.5 |
| | 11.0 | 10.20 | 1.14 | 760 | 83434 | 0.15 | 0.37 | 24.6 | 30.4 | 7.5 |
| | 12.0 | 1.02 | 0.11 | 740 | 83434 | 0.14 | 0.36 | 24.5 | 30.3 | 7.4 |
| | 16.0 | 1.02 | 0.11 | 720 | 83434 | 0.14 | 0.35 | 24.4 | 30.1 | 7.3 |
| | 20.5 | 1.02 | 0.11 | 700 | 83434 | 0.14 | 0.34 | 24.3 | 29.6 | 7.2 |

Flow 10.7 NL/h;
Conversion O₂ > 99.7%

### Example 3

22.6 mg of Catalyst A was placed in a 48.5 cm long quartz reactor with an ID of 1.0 mm. The 3.5 cm catalyst bed was located in the top part of the isothermal temperature zone of the reactor and held in place with quartz wool. Quartz inserts were not applied.

A mixture of methane (5.0 NL/h), oxygen (1.2 NL/h) and nitrogen (4.5 NL/h) at 0.96-0.98 MPa pressure was passed down flow over the catalyst at a constant temperature of 700° C. GC analysis of the product mixture, as described above, allowed the conversion of methane and oxygen and product selectivity and yield to be determined.

Key data for this experiment is detailed in Table 2.

### Comparison Example 3

This experiment was performed in a similar fashion to Example 3 using the same intake (22.6 mg) of Catalyst A in a 1 mm ID reactor, and the same pressure and temperature, but with lower gas flow: methane (2.1 NL/h), oxygen (0.53 NL/h) and nitrogen (1.7 NL/h).

Key data for this experiment is given in Table 2.

As is summarized in Table 2 below, performed at comparable GSHV the C2+ selectivity and yield in Example 3 were much higher than in Comparison Example 3, again reflecting the surprising benefits of applying higher gas linear velocities both above the catalyst bed and through the catalyst bed when OCM is performed at high partial pressures of oxygen.

TABLE 2

| | | | | | | Linear | Linear | | | |
|---|---|---|---|---|---|---|---|---|---|---|
| | | | | | | gas | gas | | | |
| | | | | | | velocity | velocity | | | |
| | | | | | | above | through | | | |
| | Run | Pressure | Pressure | | | catalyst | catalyst | Conversion | Selectivity | Yield |
| | time | Total | O₂ | Temp | GHSV | bed | bed | CH₄ | O₂₊ | O₂₊ |
| Example | (h) | (MPa) | (MPa) | (° C.) | (h⁻¹) | (m/s) | (m/s) | (%) | (%) | (%) |
| Example | 1.0 | 0.98 | 0.11 | 10.7 | 340764 | 1.38 | 3.46 | 29.7 | 57.3 | 17.0 |
| 3 | 1.5 | 0.97 | 0.11 | 10.7 | 340764 | 1.40 | 3.50 | 28.9 | 58.5 | 16.9 |
| | 2.0 | 0.96 | 0.11 | 10.7 | 340764 | 1.41 | 3.51 | 28.6 | 57.6 | 16.5 |
| Comp | 0.5 | 0.98 | 0.12 | 4.33 | 157598 | 0.56 | 1.40 | 24.2 | 39.5 | 9.6 |
| Example | 1.0 | 0.98 | 0.12 | 4.33 | 157598 | 0.56 | 1.40 | 24.3 | 39.8 | 9.7 |
| 3 | 1.5 | 0.98 | 0.12 | 4.33 | 157598 | 0.56 | 1.40 | 24.4 | 39.2 | 9.6 |
| | 2.0 | 0.98 | 0.12 | 4.33 | 157598 | 0.56 | 1.40 | 24.2 | 38.6 | 9.3 |

Temp 700° C.,
Conversion O₂ > 98.2%

### Example 4

This experiment was performed in a similar fashion to Example 1, except that the methane:oxygen ratio was varied under “undiluted” conditions.

51.3 mg Catalyst A was placed in a 48.5 cm long quartz reactor with an ID of 2.1 mm, such that the 3.5 cm catalyst bed was located in the top part of the isothermal temperature zone, between solid quartz tubes with an OD of 1.95 mm.

A mixture of methane (9.0 NL/h), oxygen (1.4-1.8 NL/h) and nitrogen (0.0-0.4 NL/h), with total flow maintained at 10.8 NL/h, and total pressure 1.0 MPa, was passed down flow over the catalyst.

The temperature was maintained at 700° C.

Key data for this experiment is given in Table 3.

As is summarized in Table 3 below, the data from Example 4 shows that application of high linear gas velocities both above the catalyst bed and through catalyst bed surprisingly leads to high C2+ selectivity and yield, even under “undiluted” conditions (feed consisting largely of methane and oxygen). The C2+ selectivity may be further increased by increasing the methane:oxygen ratio, which also results in a decrease in methane conversion.

TABLE 3

| | | | | | | Linear | Linear | | | |
|---|---|---|---|---|---|---|---|---|---|---|
| | | | | | | gas | gas | | | |
| | | | | | | velocity | velocity | | | |
| | | | | | | above | through | | | |
| | Run | Pressure | Pressure | | | catalyst | catalyst | Conversion | Selectivity | Yield |
| | time | Total | | CH₄/O₂ | GHSV | bed | bed | CH₄ | C₂₊ | C₂₊ |
| Example | (h) | (MPa) | (MPa) | ratio | (h⁻¹) | (m/s) | (m/s) | (%) | (%) | (%) |
| Example | 1.0 | 1.00 | 0.13 | 6.4 | 89135 | 2.25 | 0.78 | 19.2 | 62.2 | 11.9 |
| 4 | 6.5 | 1.00 | 0.15 | 5.6 | 89135 | 2.25 | 0.78 | 20.9 | 58.8 | 12.3 |
| | 8.5 | 1.00 | 0.17 | 5.0 | 89135 | 2.25 | 0.78 | 22.4 | 54.5 | 12.2 |
| | 11.0 | 1.00 | 0.13 | 6.4 | 89135 | 2.25 | 0.78 | 19.1 | 62.4 | 11.9 |

Temp 700° C.;
Flow 10.8 NL/h;
Conversion O₂ > 99.8%

### Comparison Example 4

The OCM performance of Catalyst A was evaluated at lower pressure, following the general procedure described above.

55 mg of Catalyst A was placed in a 48.5 cm long quartz reactor with an ID of 2.1 mm. The 4 cm catalyst bed was located in the top part of the isothermal temperature zone of the reactor. Quartz insert tubes were not applied.

A mixture of methane (5.0 NL/h), oxygen (1.2 NL/h) and nitrogen (4.5 NL/h) at 0.59 MPa total pressure was passed down flow over the catalyst. The was then reduced to methane (2.5 NL/h), oxygen (0.6 NL/h) and nitrogen (2.25 NL/h) at 0.55 MPa total pressure and temperature of 720° C., resulting in lower gas velocity.

Key data for this experiment is given in Table 4.

### Comparison Example 5

This experiment was performed using a 2.0 mm ID quartz reactor at 720° C., in a similar fashion to Comparison Example 5, except that the catalyst intake was 84.3 mg and 1.95 mm ID quartz inserts were used above and below the 7 cm catalyst bed.

The flow of gas was varied in the range, methane (2.5-7.5 NL/h), oxygen (0.6-1.8 NL/h) and nitrogen (2.25-6.75 NL/h), whilst keeping the CH₄/O₂/N₂ molar ratio constant and the pressure in the range 0.59-0.61 MPa, as detailed in Table 4. As is summarized below in Table 4, the data from Comparison Examples 4 and 5 shows that high C2+ selectivity and yields are obtained at lower oxygen partial pressures.

The examples herein that are in accordance with the present invention demonstrate that high C2+ selectivity and yields can also surprisingly be achieved at high oxygen partial pressures by employing high linear gas velocity both above the catalyst bed and through the catalyst bed.

TABLE 4

| | | | | | | Linear | Linear | | | |
|---|---|---|---|---|---|---|---|---|---|---|
| | | | | | | gas | gas | | | |
| | | | | | | velocity | velocity | | | |
| | | | | | | above | through | | | |
| | Run | Pressure | Pressure | | | catalyst | catalyst | Conversion | Selectivity | Yield |
| | time | Total | O₂ | Flow | GHSV | bed | bed | CH₄ | O₂₊ | O₂₊ |
| Example | (h) | (MPa) | (MPa) | (NL/h) | (h⁻¹) | (m/s) | (m/s) | (%) | (%) | (%) |
| Comp | 2.0 | 0.59 | 0.066 | 10.7 | 77271 | 0.54 | 1.33 | 29.0 | 63.3 | 18.4 |
| Example | 5.0 | 0.55 | 0.062 | 5.35 | 38635 | 0.28 | 0.71 | 25.5 | 54.0 | 13.8 |
| 4 | | | | | | | | | | |
| Comp | 1.0 | 0.61 | 0.066 | 5.2 | 23658 | 0.91 | 0.28 | 25.4 | 60.5 | 15.4 |
| Example | 1.6 | 0.59 | 0.062 | 10.7 | 48681 | 1.95 | 0.61 | 27.1 | 61.9 | 16.8 |
| 5 | 2.9 | 0.59 | 0.066 | 16.05 | 73021 | 2.93 | 0.91 | 27.5 | 62.5 | 17.2 |
| | 3.8 | 0.60 | 0.062 | 10.7 | 48681 | 1.93 | 0.60 | 27.4 | 60.5 | 16.6 |

Temp 720° C.,
Conversion O₂ > 98.6%

1. A process for the oxidative coupling of methane comprising converting methane to one or more C2+ hydrocarbons in a reactor, wherein said process comprises contacting a reactor feed comprising methane and oxygen with a catalyst composition and wherein the linear gas velocity of said reactor feed in the region above the catalyst bed is at least 0.6 m/s, the linear gas velocity through the catalyst bed is at least 0.6 m/s and the partial pressure of oxygen in the reactor is greater than 0.08 MPa.

2. The process according to claim 1, wherein the total pressure in the reactor is greater than 0.6 MPa.

3. The process according to claim 1, wherein the linear gas velocity of the reactor feed in the region above the catalyst bed is at least 0.65 m/s.

4. The process according to claim 1, wherein the linear gas velocity of the reactor feed through the catalyst bed is at least 0.65 m/s.

5. The process according to claim 1, wherein the region above the catalyst bed comprises inert porous packing.

6. The process according to claim 1, wherein the partial pressure of oxygen in the reactor is greater than 0.1 MPa.

7. The process according to claim 1, wherein the gas hourly space velocity is in the range of from 30000 to 3000000 h⁻¹.

8. The process according to claim 1, where the conversion is carried out at a reactor temperature in the range of from 650 to 850° C.

9. The process according to claim 1, wherein the catalyst composition comprises manganese, one or more alkali metals and tungsten on a carrier.

10. The process according claim 1, wherein the catalyst composition comprises manganese, sodium and tungsten on a silica carrier.
